(19) United States
(12) Patent Application Publication
(10) Pub. No.: US 2004/0199232 A1
(43) Pub. Date: Oct. 7, 2004

(54) Feedback device having electrically conductive fabric

(76) Inventors: Gordon George Wallace, New South Wales (AU); Peter Charles Innis, New South Wales (AU); Julie Robyn Steele, New South Wales (AU); Dezhi Zhou, New South Wales (AU); Geoffrey Maxwell Spinks, New South Wales (AU)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/486,481

(22) PCT Filed: Aug. 9, 2002
(86) PCT No.: PCT/AU02/01074

(30) Foreign Application Priority Data
Aug. 10, 2001 (AU) PR 6944

(51) Int. Cl. A61N 1/04

(52) U.S. Cl. 607/115

(57) ABSTRACT

A feedback device for detecting a mechanical input and providing a feedback indication. For example, a wearable biomechanical feedback device that has an electrically conductive fabric sensor that can be closely fitted to a limb. Movement of the limb causes strain on the fabric sensor which alters its electrical impedance and triggers the feedback indication.

### FIELD OF THE INVENTION

The present invention relates to devices for monitoring the motion of structures and/or forces on and within moving structures. The invention has particular application to a device for immediate biofeedback in response to the motion and/or forces generated by the moving structures.

### BACKGROUND OF THE INVENTION

Within the discipline of biomechanics , the primary interest of researchers is focused on examining the forces acting upon and within biological structures as well as the effects produced by these forces. In this field many principles are drawn from related disciplines such as anatomy, psychology, mathematics, physics and mechanics. These principles are used to gain a better understanding of the biological effects of forces on living tissues, growth and development, overload and injury, and other factors affecting the movement of body segments. Biomechanics therefore has application in a diverse range of occupations including orthopaedic surgery, exercise rehabilitation, ergonomics, biomedical engineering as well as coaching and teaching sports skills.

The invention is disclosed herein with particular reference to its application as a sports training tool and rehabilitation aid. However, it will be immediately apparent to those of ordinary skill in this field that the present invention is readily applicable to many other uses and applications. For example, it may be easily adapted for use as means for generating input signals for controlling a device such as a computer. It could also be adapted for many different types of amusement novelties or playthings.

Within the arena of sport, biomechanics has numerous applications including:

identifying techniques to optimise sports performance while minimising the risk of injury to the performer;

evaluating the effects of rule modifications on player safety and performance; and

developing appropriate sports equipment both to enhance performance and to protect athletes. This equipment includes items required to participate in a sport, such as sporting implements as well as clothing which is suitable for the athlete to perform the required skills.

To better understand the motion of segments and the forces acting on the human body during movement, biomechanists employ a variety of quantitative techniques. These include electromyographic devices to measure muscle activity, cine/video or optoelectronic devices to quantify the external motion of an athletes body segments and force platforms or other dynamometry devices to measure the forces generated during a performance. Information from these devices is combined with data describing the dimensions of an athlete's body segments to mathematically model the forces generated during a performance and the effects of these forces on the athlete's body.

Although advances in technology have provided highly sophisticated apparatus for biomechanical analysis of human performance, some restrictions and disadvantages exist. For example, many of the devices that are attached to an athlete's body during a performance analysis have rigid components, which do not conform to the athlete's body shape. This will tend to interfere with their natural motion during a performance. Other devices have an overly restricted operational range. For example, traditional strain gauges are typically restricted to operating over a dynamic range of approximately 10% (that is a 10% variation in the length of the operational sections of the strain gauge).

### SUMMARY OF THE INVENTION

It is an object of the present invention to overcome or ameliorate at least one of the disadvantages of the prior art, or to provide a useful alternative.

According to a first aspect, the present invention provides a feedback device for a structure, the device including:

electrically conductive fabric for establishing an electrical current path with an electrical impedance, such that mechanical input to the device causes a change to the electrical impedance;

a voltage source to cause current to flow along the current path; and

a sensor for detecting change in the electrical impedance of the current path and producing an immediate feedback indication in response to a predetermined mechanical input to the device.

According to a second aspect, the present invention provides a method for producing a feedback indication in response to a mechanical input to a device, the method including:

attaching electrically conductive fabric to the structure, the fabric having an electrical current path position on the structure such that a predetermined mechanical input to the structure causes the electrical impedance associated with the current path to change;

applying a voltage across the current path; and

using a sensor for detecting the change in the impedance and producing an immediate feedback indication.

Preferably, the device is a biomechanical feedback device and the structure is a moveable biological structure. In another preferred form, the electrically conductive fabric is an elastic fabric at least partially coated with an electrically conductive polymer material.

Preferably, the elastic fabric is formed for close fitting to the biological structure and movement therewith. In a particularly preferred form, the fabric is a synthetic fabric such as that marketed under the trade mark “lycra™. Please note that “lycra™” is the registered trade mark of E I Du Pont De Nemours and Company. However, the elastic fabric may also include other suitably elastic fabrics such as wool or polyester. Alternatively, the electrically conductive fabric is a metallated fabric, carbon loaded fabric or other suitable fabric incorporating a flexible strain transducer element.

In some preferred embodiments, the feedback indication is an audio signal that can be heard by the user, however, in other forms the indication is a vibration or other mechanical input that is sensed by the user. The feedback indication may also be a change in colour of part of the sensor. In these embodiments, the sensor may include a transparent electrode coated with an inherently conductive polymer having a colour that is dependent on its oxidation state such that oxidation or reduction caused by current changes resulting from the mechanical input will produce a visible colour change. Preferably, the inherently conductive polymer is a polypyrrole, a polythiophene or a polyaniline. In a further preferred form, the transparent electrode is formed from indium tin oxide.

The feedback indication may also be in the form of a taste or smell.

In some forms, the sensor includes a transducer and separate feedback indicator. Still further embodiments of the sensor include a transmitter to allow the feedback indicator to be remotely positioned. Typically, the transmitter is configured to send signals in the microwave frequency range although any other frequency would be suitable. These embodiments allow the feedback indicator to be a remotely positioned computer screen or analogue/digital display.

Preferably, the sensor includes a wheatstone bridge circuit where the electrical current path provided by the conductive polymer material is the variable resistance segment of the circuit.

In one form, the electrical current path provided by the conductive polymer material is a strip coated on the elastic fabric such that the length of the strip aligns with the direction of extension of the elastic fabric caused by the mechanical input of interest. In other preferred forms, the conductive polymer material is coated on the elastic fabric in a U-shaped configuration such that the sides of the U align with the direction of extension of the elastic fabric caused by the mechanical input of interest. This allows electrodes at either end of the strip to be positioned relatively close together so that the overall design is relatively compact.

In a further preferred form, the conductive polymer material is coated on the elastic fabric in a multi-pronged fork configuration wherein the length of each prong aligns with the direction of extension in the elastic fabric caused by the mechanical input of interest. The multi-pronged configuration allows the prongs to have different inherent electrical resistance characteristics and/or different dynamic ranges. Hence, the electrodes can be attached to the ends of any selected pair of prongs for different response characteristics.

In some embodiments, the feedback indication is produced whenever a mechanical input is greater than a predetermined threshold. In the alternative, the indication could be produced whenever a mechanical input is less than a predetermined threshold. In a preferred embodiment, the device includes two or more current paths, such that the feedback indication from a first current path is produced by a mechanical input greater than a first threshold and the feedback indication from a second current path is produced by a mechanical input greater than a second threshold. This allows the device to track the location and movement of the mechanical input or inputs to the device.

In a still further preferred form, the first threshold is different to the second threshold. A multi-strip sensor enables simple direct bio-feedback on the rate of movement of a biological structure. For example, if a first strip triggers the feedback indication at a threshold of 20% extension and a second strip triggers the feedback indication at 40% extension, the time between triggers can be used to derive the appropriate rate.

In one form, the first and second current paths are closely adjacent. This configuration can record a localised movement rate and in other forms, the first and second current paths are on separate locations of the biological structure. With the strips on different parts of the body and with the use of telemetry, the device can provide a complex and accurate analysis of a body's motion.

In a particularly preferred form, the sensor has two or more current paths in a laminated structure. In this form, each of the current paths trigger a feedback indication at different thresholds. ln one form, the laminated structure has layers including different polymer coatings, each coating forming one of the current paths, wherein each coating produces a feedback indication at different degrees of extension. Using the laminated structure, the sensor can have a greater operational. By including a fabric with appropriate mechanical recoil characteristics in the laminate (not necessarily the sensing fabric), the response times and fabric recoil are improved.

In another embodiment, the current path has non-uniform conductivity characteristics along its length whereby the sensor can detect the changes in impedance of predetermined sections of the current path such that each section triggers a feedback indication at different thresholds of mechanical input. The use of a graded resistance strip in the sensor can also increase the linear dynamic range of the device.

In some forms of the present invention, the mechanical input of interest is pressure applied to the current path. In a preferred form, the current path is provided by a laminated assembly including a fabric layer sandwiched between two polymer layers.

In some embodiments, the device is configured to monitor lower limb motion, and in particular, monitoring knee joint motion and/or ankle joint motion. In a particularly preferred embodiment, the device is used as a training aid during landing training programs for participants in sports with a high incidence of knee and ankle injuries such as football, netball, basketball or skiing. In other embodiments, the device is configured to monitor upper limb motion. In some forms of these embodiments the device is used as a training aid to improve the bowling technique of a cricketer, improve the basket shooting technique of a basketballer or netballer, improve the serving technique for a tennis player or improve the swing of a golfer. Preferably, the fabric is formed into a sleeve wherein the conductive polymer coating is positioned on the sleeve, such that in use, the feedback indicator provides an indication in the form of an audio signal to alert the participant when they are using inappropriate limb joint motion. Of course, the indicator couldjust as easily produce an indication in response to an appropriate limb joint motion.

The present invention allows the production of a wearable system that utilises the changes in resistance of a polymer with a high degree of flexibility and elasticity as a trigger to provide immediate feedback as to whether a movement is being performed correctly or not. This allows the participant to instantly adjust and correct their technique. Hence, the correct technique is quickly learnt and reinforced during the activity. This forms the basis of a highly effective training or rehabilitation program which may be customised and optimised to suit particular activities and participants.

### Brief Description of the Drawings

Preferred embodiments of the present invention will now be described by way of example only with reference to the drawings in which:

FIG. 1 shows a schematic representation of a device according to the present invention;

FIG. 2a shows a schematic representation of equipment used to test conductive polymer coated fabric for use in the present invention;

FIG. 2b is a graph showing the results of tests conducted on the equipment shown in FIG. 1;

FIG. 3 shows the circuit diagram of a more sophisticated form of the present invention;

FIG. 4a is a schematic representation of a U-shaped feedback device; FIG. 4b is a schematic representation of a feedback device with a multi-pronged configuration;

FIG. 5 is a schematic representation of a feedback device with a multi-strip sensor in accordance with the present invention;

FIG. 6a is a schematic representation of the different polymer coating layers in a laminated sensor for a feedback device according to the present invention;

FIG. 6b is a graphical representation of the resistance versus the extension for the different polymer coatings of FIG. 6a;

FIG. 7a is a schematic representation of a feedback device configured for the measurement of pressure;

FIG. 7b shows a schematic representation of a laminated structure to be used for the measurement of pressure; and

FIG. 8 is a schematic sectional view of a feedback device according to the present invention which provides a feedback indication form of the colour change.

### DETAILED DESCRIPTION OF PREFERRED EMBODIMENTS

It has recently been shown (see De Rossi, D., Della Santa, A., Mazzoldi, A. Material Science Engineering. 1999, C7, 31, the contents of which are incorporated herein by cross-reference), that conducting polymer coated lycra™fabrics can function as strain gauges with minimal changes to the fabric properties incurred after coating. The development of certain embodiments of the present invention has now shown that using appropriate polymerisation conditions or different host fabric structures can modify the dynamic range of these fabric strain gauges.

### EXAMPLE PREPARATION OF SENSING STRIPS

Reagents

Monomer: Pyrrole (Aldrich): 0.015 M or 1 ml/500 ml H₂O

Dopant: NDSA (Aldrich): 1,5-Naphthalenedisulfonic acid tetrahydrate 0.005 M or 1.8 gm/500 ml H₂O

Oxidant: FeCI₃ (BDH): Ferric chloride 0.04 M or 25.98 gm/L H₂O

Procedure

NDSA and pyrrole are dissolved in water (500 ml). Lycra fabric (4 strips −2 cm×25 cm) are sewn onto wire racks and soaked in the monomer/dopant solution (20 mins). The solution is clear in colour.

An aliquot of solution (250 ml) is then decanted and an aliquot of FeCI₃ (250 ml) added. Initially this solution is yellow.

The pyrrole in solution begins to be oxidized, after approximately 3 mins, by the FeCl₃. The solution turns a green colour.

Oxidation continues and after 5-6 mins the solution begins to darken to a black colour.

As oxidation continues further polypyrrole powder settles out of the solution. The process continues until the coated fabric is removed from the solution.

After 70 mins the lycra strips are coated with a thin layer of polypyrrole and have changed from a white colour to a black colour.

The strips are dried, removed from the wire rack and washed in several changes of water for 45 mins to remove any residual polypyrrole powder not coated onto the surface of the fabric. The dried washed strips are ready for use.

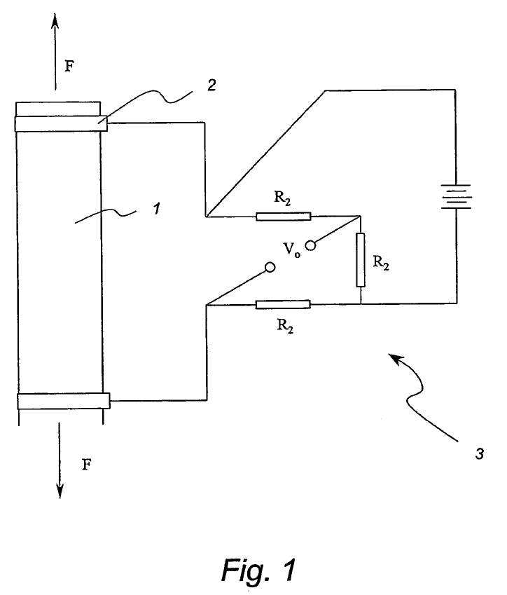

Using the set up shown in FIG. 1, calibration curves for different polyrnerisation conditions or different host fabrics may be generated. The electrically conductive polymer coated fabric 1 is held under tension between copper foil strips 2. Applying a force F to the ends of the fabric 1 increases the tensile load and the resultant strain in the fabric causes it to elongate. The elongation of the fabric 1 in turn causes the electrical resistance of the polymer coating structure tochange. The change in resistance associated with the strain caused by force F can be measured using a simple wheatstone bridge arrangement 3 and a voltage sourceV. Ordinary workers in this field will readily understand the operation of a wheatstone bridge whereby a potentiometer Vo measures the change in potential difference between the points shown in FIG. 1. Hence, from the potentiometer V₀ and the known resistance of the resistors R₂, this simple set up can be used to generate a set of calibration curves showing the resistance versus strain for various types of fabric and polymerisation conditions. Using these curves, the feedback device can accurately equate electrical resistance with particular movements of a biological structure. It will also be readily appreciated that it would be possible to use many other types of comparitor circuits instead of the wheatstone bridge.

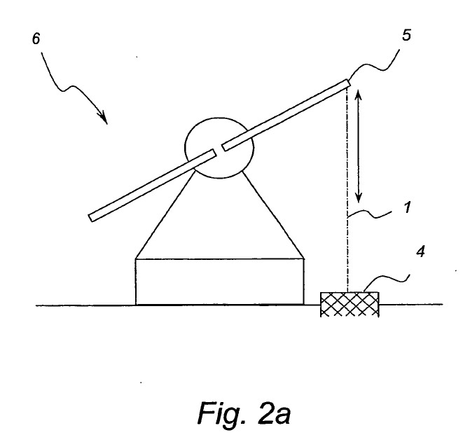

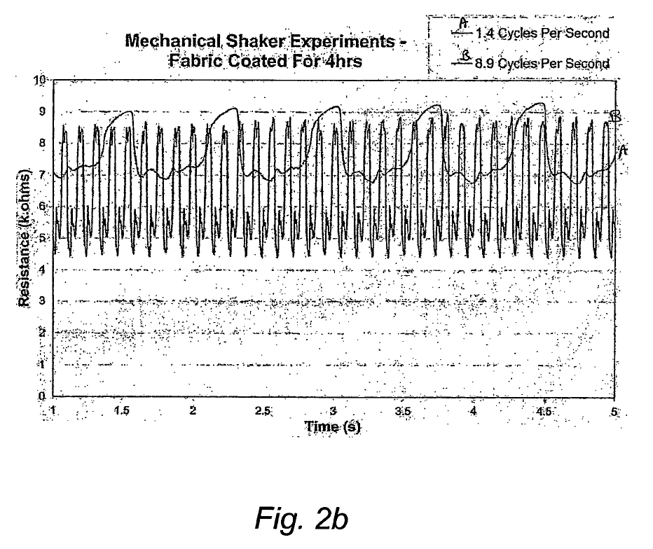

To better simulate the dynamic environment provided by athletes' limbs during sporting manoeuvres, the conductive polymer coated fabrics were tested with an arrangement shown in FIG. 2a. The polymer coated fabric 1 is held between a fixed clamp 4 and end of an oscillating arm 5 of a mechanical shaker 6. Using the same wheatstone bridge arrangement, responses, such as that shown in FIG. 2b, may be generated for various fabrics and polymerisation conditions, over a range of frequencies. Using these responses, it is possible to produce a feedback device in the form of a knee sleeve. Configuring the knee sleeve to provide feedback whenever inappropriate motion of the knee occurs, athletes can be given immediate feedback during training. For example, learning to land correctly can help to protect Australian Rules Football (ARF) players against knee injury, in particular against non-contact anterior cruciate ligament (ACL) injury. The knee sleeve form of the present invention is a simple inexpensive sleeve containing a strip or strips of elastic fabric fully or partially coated with an electrically conductive polymer and an electrical circuit configured to emit an audio signal in response to predetermined movements.

The human knee joint has a high susceptibility to injury due to its incongruent structured and the high forces imposed on the joint, particularly during dynamic activities such as landing. Although knee injuries account for only 12% of total sport injuries, in Australia they represent approximately 25% of total injury costs. It has been estimated that the direct cost of knee injuries in sport per year was as high as $11.9 million dollars for Rugby League and Rugby Union, and $8.8 million dollars for Australian Rules Football. These costs have continued to escalate over the past decade.

Of all the knee ligaments, the ACL is the most frequently injured with an injury frequency 9 times greater than that of the posterior cruciate ligament. Rupture of the ACL is also one of the most debilitating injuries ARF player's can sustain, especially the younger players. When the native ACL is ruptured, the knee joint is predisposed to episodes of “giving way”, further risk of meniscal damage, loss of proprioception via damage to the mechanoreceptors in the joint and ligament itself, recurrent pain, and likely degeneration of the knee joint as a result of excessive laxity and persistent instability.

Mechanisms of ACL injury in sport can be classified into two main categories:

a) contact injury is caused when an external force is applied to the knee causing ACL rupture; and

b) non-contact injuries caused when the indirect force is applied to the knee.

Typically non-contact ACL injury involves rapid deceleration, quick changes in direction, and or abrupt landings, often accompanied by poor landing technique. It has been estimated that 66% to 78% of ACL injuries occur via non contact mechanisms.

Whereas contact injuries have mainly been attributed to chance, non contact ACL injuries are more related to characteristics of the injured individual such as the degree of muscular weakness or muscular coordination and therefore the movement pattern performed at the time of injury. Where poor landing technique is displayed, it would appear feasible to prevent non contact ACL injuries by correcting this technique. In light of this, strategies are urgently needed by which players can learn to land correctly.

It has been shown that giving verbal feedback to subjects before they performed a vertical drop jump resulted in the subjects generating less ground reaction force upon landing. Subjects were able to quickly and effectively assimilate verbal instructions so as to modify their lower limb movement patterns to generate less force upon ground impact. Based on this reduction in ground reaction forces it was suggested that subjects were able to be trained to modify their landing technique to reduce their risk of injury. The benefits of landing programs in reducing knee injuries in ARF have also been acknowledged by the implementation of landing training programs for ARF players.

Extensive biomechanical research has also shown that flexing the knees throughout the landing action can “cushion” the forces over a longer time and thereby dissipate the shock loading of landing. Increased knee flexion also lowers a player's centre of gravity which in turn enhances their stability. To reduce knee injury at landing a relatively high flexion angle should be combined with a large range or amplitude of joint motion to dissipate the energy in muscles.

The knee sleeve can be used to train players to land correctly, so that they flex their knees through a desirable range of motion throughout a landing action which in turn reduces the risk of injury. The knee sleeve would have the advantage of providing immediate individualised feedback to any player wearing the device during a training program. This improves the objectivity, frequency and speed of feedback provided to players about their landing technique.

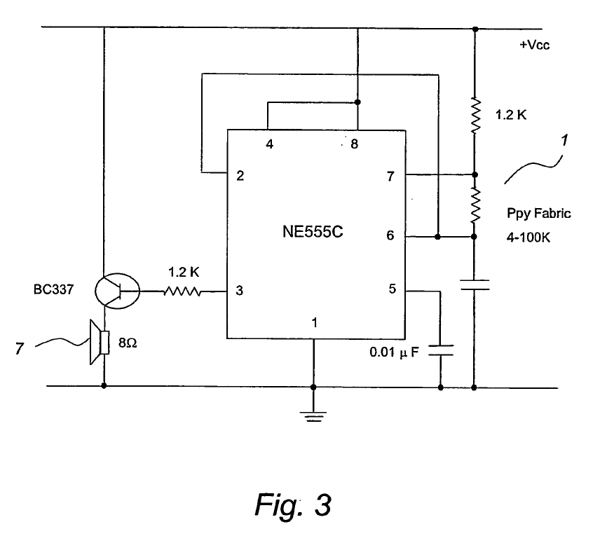

Referring to FIG. 3, the active component of the conducting polymer coated lycra strain gauge 1 has been incorporated into a wearable electronics circuit. The fabric 1 is part of an electronic circuit whereby if the knee flexion angle during landing is insufficient or too great an audio signal 7 will be emitted. The range of knee flexion angles at which the audio tone is emitted can be varied. This audio signal 7 provides immediate feedback to the wearer allowing them to adjust their knee landing technique accordingly. All components, including the audio alarm 7 , are enclosed within the knee sleeve itself without the need for any external components to allow the system to function.

This arrangement is inexpensive and extremely light weight, as it is not significantly heavier than a standard elastic knee sleeve. Accordingly, the knee sleeve will not be an impediment to normal movement during landing training. As the sleeve is made from flexible fabric and containing minimal rigid components around the knee it is safe in contact situations.

In one version of the knee sleeve, the polymer coating on the fabric is a strip positioned so that it runs down the anterior aspect of the thigh, knee, and leg. This sleeve is constructed to emit an audio tone when the knee flexion angle (sagittal plane only), reaches a set threshold. Angle changes are defected by lengthening the polymer coated strip as the knee flexes and extends. The threshold at which the tone is emitted can be varied. The sleeve is a robust device for use in training sessions that can provide highly consistent and accurate feedback by the audio tone.

Athletes in sports in which non-contact ACL ruptures are caused by abrupt deceleration without tibial rotation are frequent. The coaches and trainers of elite athletes involved in these sports generally have high levels of expertise and can configure and incorporate this version of the knee sleeve into their existing training programs. Similarly, clinicians such as physiotherapists can use this version of the device to provide patients with feedback during rehabilitation exercises.

Another version of the knee sleeve can include telemetry so that knee angle data can be recorded on computer in real time for later analysis. Of course in this version, the audio tone would be a secondary indicator and therefore optional. This refined version is a field instrument to measure knee angle during movement activities and possibly during games and training as well. A microwave transmitter sends the data to the remotely positioned receiver where it is stored in real time. This version is a particularly useful biomechanical measurement tool suitable for any of the above mentioned sports as well as for field testing of sports such as alpine skiing.

For participants in sports who do not have access to trainers with expertise in designing training programs, the knee sleeve may be marketed with some type of interactive multi-media. For instance, the sleeve may be sold with a CD to guide the players through appropriate activities to use the sleeve and improve their techniques. Of course clinicians can also provide patients with take home instructions in some form of multi-media to help speed up their rehabilitation.

In a still further modification, additional polymer strips can be provided to enable monitoring of the rotation of the leg relative to the thigh (in addition to monitoring knee flexion). Once again, the sleeve can emit feedback via an audio tone, vibration or storage of the data. The ability to monitor tibial rotation in addition to knee flexion would be more appropriate in sports and activities in which the non-contact ACL rupture mechanism involves tibial rotation as well as deceleration. Such movements are typical during side stepping manoeuvres in soccer and the rugby codes.

The knee sleeve versions of the present invention discussed above are purely illustrative. Skilled workers in this field will readily recognise many other applications and embodiments that use a wearable sensor. These include embodiments such as:

use of the knee sleeve in training footballers to kick “through” the ball;

to monitor head, torso and or limb motion to teach correct posture during activities of daily living, work and recreation;

to monitor elbow motion during bowling training in cricket to detect an illegal bowling action involving excessive elbow flexion and providing the bowler with feedback in order to correct their technique;

to monitor torso motion using a wearable vest with arrays of fabric strain gauges incorporated to be used in training cricketers to bowl;

to monitor wrist motion during basketball or netball shooting practise in order to detect if the hand is deviating medially or laterally;

monitoring elbow and/or wrist motion during a tennis serve;

monitoring the torso, wrist, elbow and/or knee movements during golf swings;

configuring the invention so that the trigger point can be incrementally increased or decreased to reflect the progressive increasing of performance (degrees of movements) of a patient during a rehabilitation program;

configuring the device into a form such as a glove in order to allow a person to generate the input signals for a computer or other equipment; and

the invention may be incorporated into toys or other playthings in order to provide a response to certain interactions with the child.

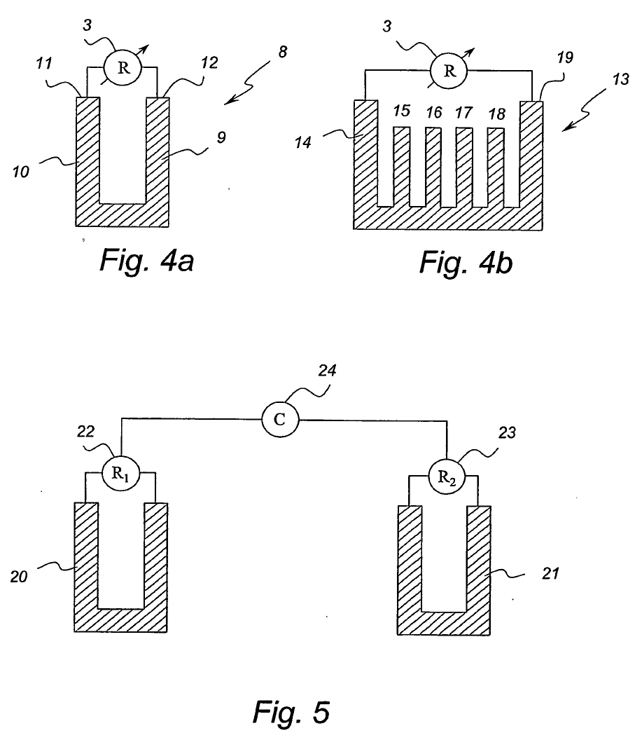

FIGS. 4 to 8 show various embodiments and refinements of the more fundamental design. FIG. 4a shows the current path provided by the element coating formed into a U-shaped configuration. The sides 9 and 10 of the U shape are aligned with the direction of extension in the underlying fabric caused by the movement of interest. In this configuration, the ends of the current path 11 and 12 are closer together which simplifies the connection of the electronics 3 and allows for a more compact design.

In FIG. 4, the conductive polymer coating is formed into a multi-pronged configuration 13. Each of the prongs 14 to 19 are aligned with the direction of extension in the underlying fabric caused by the movement to be monitored. Each prong can be designed to have a different conductivity so that the electronics 3 can be attached to any selected pair of prongs in order to change the response characteristics of the sensor.

The feedback device shown in FIG. 5 incorporates multiple sensors. Two separate U-shaped strips 20 and 21 are connected to respective wheatstone bridge circuits 22 and 23. In turn, the wheatstone bridges 22 and 23 are linked to a combined comparator and signal generator 24. By designing the strips so that they trigger a feedback indication at different thresholds of strain, the multi-strip sensor can provide simple and direct bio-feedback on the rate of movement in a biological structure. For example, the strip 20 may trigger a feedback indication at a threshold of 20% strain whereas the strip 21 triggers at a threshold of 40% strain. By monitoring the time between triggers, the rate of movement can be derived. Accordingly, the use of a network of differentially triggered sensors positioned on a range of body parts coupled with suitable telemetry can provide a complex analysis of its motion.

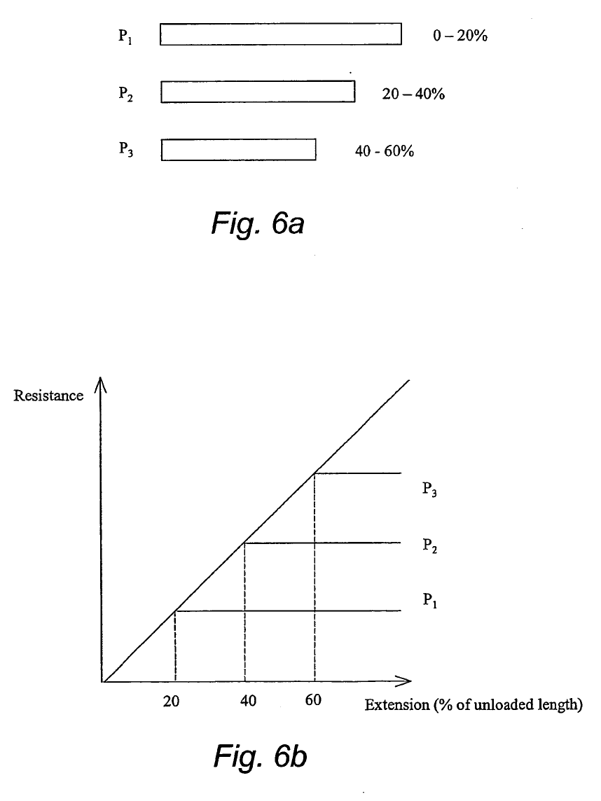

The use of a laminated structure enables a device to get a better linear range, response time as well as quicker textile recall. FIG. 6a diagrammatically shows three polymer coatings of a laminate, each with different operative ranges. P₁ has an operative range of 20%. Hence, any extension greater than this threshold will trigger the feedback indication associated with P₁. Likewise P₂ and P₃ have operative ranges of 40% and 60% respectively whereby their feedback indications trigger at these respective thresholds. FIG. 6b presents this in a graphical format to highlight the extended operational range provided by the laminated structure.

Similarly, a polymer strip with non-uniform conductivity along its length can increase the dynamic range of the sensor. By providing sections of the strip with different conductivity and therefore different operational ranges, the sensor can provide feedback over an extended range of extension in the underlying fabric.

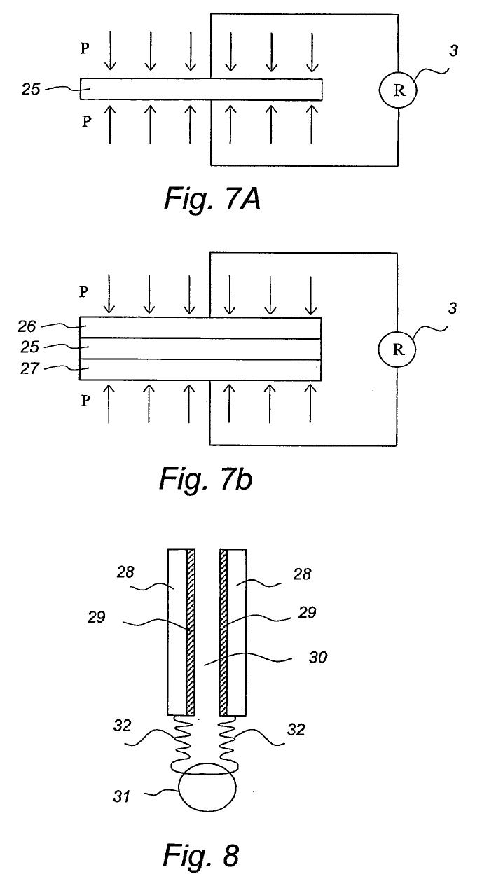

FIG. 7a is a diagrammatic representation of the sensor configured to monitor mechanical input in the form of a pressure P. The pressure P changes the conductivity of the fabric 25 by pressing its fibers closer together. This becomes the variable resistance segment of the wheatstone bridge arrangement 3.

In a further refinement, a laminated structure shown in FIG. 7b is used to monitor the pressure P. By sandwiching fabric 25 between two polymer layers 26 and 27 , the sensitivity of its conductivity to changes in pressure is increased. The conductivity of the polymer layers 26 and 27 is selected such that it is much higher than the fabric 25 so that it is the changes in resistance of the fabric which provide the threshold switch for triggering the feedback indication.

FIG. 8 shows a form of the feedback device which provides the relevant feedback indication in the form of a colour change. The colour of some inherently conductive polymers such as polypyrroles, polythiophenes and polyanilines is highly dependent on the oxidation state of the polymer. The reduction or oxidation of the polymer can dramatically changes its UV-visible absorption characteristics. For polythiophene, the process is represented by the equation shown below:

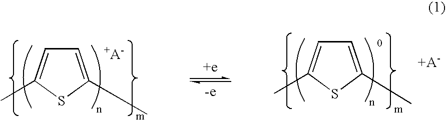

As shown in FIG. 8, the polymer 28 is coated onto a support electrode 29. One suitable electrode material is indium tin oxide. Between the electrodes 29 is a suitable electrolyte 30, the coated electrodes 29 are connected to a voltage source 31 via the fabric strain sensor 32. Changes in the resistance of the fabric strain sensor caused by the movement to be monitored will result in a corresponding change to the current through the circuit. This current change can be used to trigger the colour change in the polymer 28. Hence, the colour change provides the user with an immediate and direct feedback indication of the threshold movement of interest.

The present invention has been described herein by way of example only. The various embodiments are entirely illustrative and in no way restrictive on the spirit and scope of the broad inventive concept. The findamental principles and background technology employed in various aspects of the present invention is comprehensively set out in the following references, which are incorporated herein by cross-reference:

1. “Chemistry and Electrochemistry of Conducting Polymers: —A Handbook for Smart Materials Scientists” Wallace, G. G., Teasdale, P. R., Spinks, G., Technomic Publ. Co., Lancaster, 1997.

2. Dressware:wearable hardware” D. DeRossi et al. —Materials Science and Engineering, 1999, C 7,31-35

3. “Conductive Textiles” R. V. Gregory et al -Synthetic Metals 1989,28, C823-C835

4. “Characterisation and Application of polypyrrole coated Textiles”-H.Kuhn in Inherently Conducting Polymers:An Emeerging Technology. M.Aldissi (Ed).

Kluwer Publishers, 1993 p. 25

1. A feedback device for a structure, the device including:
electrically conductive fabric for establishing an electrical current path with an electrical impedance, such that mechanical input to the device causes a change to the electrical impedance;
a voltage source to cause current to flow along the current path; and
a sensor for detecting change in the electrical impedance of the current path and producing a feedback indication when mechanical input to the device occurs.

2. A feedback device according to claim 1, wherein the device is a biomechanical feedback device and the structure is a moveable biological structure.

3. A feedback device according to claim 1, wherein the structure is a piece of sporting equipment such as a racquet or a club.

4. A feedback device according to claim 2, wherein the electrically conductive fabric is an elastic fabric at least partially coated with an electrically conductive polymer material.

5. A feedback device according to claim 4, wherein the elastic fabric is formed for close fitting to the biological structure and movement therewith.

6. A feedback device according to claim 5, wherein the elastic fabric is a synthetic fabric such as that marketed under the trade mark “lycra™”.

7. A feedback device according to claim 5, wherein the elastic fabric is a blend such as cotton lycra or wool lycra.

8. A feedback device according to claim 5, wherein the elastic fabric is wool or polyester.

9. A feedback device according to claim 5, wherein the electrically conductive fabric is a metallated fabric, carbon loaded fabric or other suitable fabric incorporating a flexible strain transducer element.

10. A feedback device according to claim 1, wherein the feedback indication is an audio signal.

11. A feedback device according to claim 1, wherein the indication is a vibration or other mechanical stimulus that is sensed by the user.

12. A feedback device according to claim 1, wherein the feedback indication is a change in colour of part of the sensor.

13. A feedback device according to claim 12, wherein the sensor includes a transport electrode coated with an inherently conductive polymer having a colour that is dependent on its oxidation state such that oxidation or reduction caused by current changes resulting from the mechanical input will produce a visible colour change.

14. A feedback device according to claim 13, wherein the inherently conductive polymer is a polypyrrole, a polythiophene or a polyaniline.

15. A feedback device according to claim 13, wherein the transport electrode is formed from indium tin oxide.

16. A feedback device for a structure according to claim 1, wherein the sensor includes a transducer and separate feedback indicator.

17. A feedback device according to claim 16, wherein the sensor includes a transmitter to allow the feedback indicator to be remote from the transducer.

18. A feedback device according to claim 1, wherein the transmitter operates in the microwave frequency range.

19. A feedback device according to claim 1, wherein the sensor includes a wheatstone bridge circuit where the electrical current path provided by the conductive polymer material is the variable resistance segment of the circuit.

20. A feedback device according to claim 19, wherein the electrical current path provided by the conductive polymer material is a strip coated on the elastic fabric such that the length of the strip aligns with the direction of extension of the elastic fabric caused by the mechanical input of interest.

21. A feedback device according to claim 19, wherein the conductive polymer material is coated on the elastic fabric in a U-shaped configuration such that the sides of the U align with the direction of extension of the elastic fabric caused by the mechanical input of interest.

22. A feedback device according to claim 19, wherein the conductive polymer material is coated on the elastic fabric in a multi-pronged fork configuration wherein the length of each prong aligns with the direction of extension in the elastic fabric caused by the mechanical input of interest.

23. A feedback device according to claim 22 wherein the prongs have different inherent electrical resistance, wherein electrodes attached to the ends of any selected pair of prongs produce different response characteristics to a mechanical input.

24. A feedback device according to claim 19, wherein the feedback indication is produced whenever a mechanical input is greater than a predetermined threshold.

25. A feedback device according to claim 24, wherein the device includes two or more current paths, such that the feedback indication from a first current path is produced by a mechanical input greater than a first threshold and the feedback indication from a second current path is produced by a mechanical input greater than a second threshold.

26. A feedback device according to claim 25, wherein the first threshold is different to the second threshold.

27. A feedback device according to claim 26, wherein the first and second current paths are closely adjacent.

28. A feedback device according to claim 24, wherein the sensor has two or more current paths in a laminated structure.

29. A feedback device according to claim 28, wherein each of the current paths trigger a feedback indication at different thresholds.

30. A feedback device according to claim 29, wherein the laminated structure has layers including different polymer coatings, each coating forming one of the current paths, wherein each coating produces a feedback indication at different degrees of extension.

31. A feedback device according to claim 24, wherein the current path has non-uniform conductivity characteristics along its length whereby the sensor can detect the changes in impedance of predetermined sections of the current path such that each section triggers a feedback indication at different thresholds of mechanical input.

32. A feedback device according to claim 1, wherein the mechanical input of interest is pressure applied to the current path.

33. A feedback device according to claim 32, wherein the current path is provided by a laminated assembly including a fabric layer sandwiched between two polymer layers.

34. A feedback device according to claim 1, wherein the device is configured to monitor lower limb motion.

35. A feedback device according to claim 34, wherein the device is configured for monitoring knee joint motion and/or ankle joint motion.

36. A feedback device according to claim 35, wherein the device is used as a training aid during landing training programs for participants in sports with a high incidence of knee and ankle injuries such as football, netball, basketball or skiing.

37. A feedback device according to claim 1, wherein the device is configured to monitor upper limb motion.

38. A feedback device according to claim 1, wherein the device is configured to monitor torso, head and/or neck motion.

39. A feedback device according to claims 37, wherein the device is used as a training aid to improve the technique of participants in activities such as the bowling technique of a cricketer, improve the basket shooting technique of a basketballer or netballer, improve the serving technique for a tennis player or improve the swing of a golfer, or improve the posture of participants in activities of daily life, work or recretion.

40. A feedback device according to claim 34, wherein the fabric is formed into a sleeve wherein the conductive polymer coating is positioned on the sleeve, such that in use, the feedback indicator provides an indication in the form of an audio signal to alert the participant when they are using inappropriate limb joint motion.

41. A method for producing a feedback indication in response to a mechanical input to a structure, the method including:
attaching electrically conductive fabric to the structure, the fabric having an electrical current path position on the structure such that mechanical input to the structure causes the electrical impedance associated with the current path to change;
applying a voltage across the current path; and
using a sensor for detecting the change in the impedance and producing a feedback indication.

42. A method according to claim 41, wherein the device is a biomechanical feedback device and the structure is a moveable biological structure.

43. A method according to claim 42, wherein the electrically conductive fabric is an elastic fabric at least partially coated with an electrically conductive polymer material.

44. A method according to claim 41, wherein the feedback indication is an audio signal.

45. A method according to claim 41, wherein the indication is a vibration or other mechanical stimulus that is sensed by the user.

46. A method according to claim 41, wherein the feedback indication is a change in colour of part of the sensor.

47. A method according to claim 46, wherein the sensor includes a transport electrode coated with an inherently conductive polymer having a colour that is dependent on its oxidation state such that oxidation or reduction caused by current changes resulting from the mechanical input will produce a visible colour change.

48. A method according to claim 41, wherein the sensor includes a transducer and separate feedback indicator.

49. A method according to claim 48, wherein the sensor includes a transmitter to allow the feedback indicator to be remote from the transducer.

50. A method according to claim 41, wherein the sensor includes a wheatstone bridge circuit where the electrical current path provided by the conductive polymer material is the variable resistance segment of the circuit.

51. A method according to claim 41, wherein the feedback indication is produced whenever a mechanical input is greater than a predetermined threshold.

52. A method according to claim 41, wherein the device is configured to monitor lower limb motion.

53. A method according to claim 41, wherein the device is configured to monitor upper limb, torso, head and/or neck motion.

54. A method according to claim 52, wherein the device is configured for monitoring knee joint motion and/or ankle joint motion, and/or hip/joint motion.

55. A method according to claim 41, wherein the device is used as a training aid during landing training programs for participants in sports with a high incidence of knee and ankle injuries such as football, netball, basketball or skiing.

56. A method according to claim 52, wherein the fabric is formed into a sleeve wherein the conductive polymer coating is positioned on the sleeve, such that in use, the feedback indicator provides an indication in the form of an audio signal to alert the participant when they are using inappropriate limb joint motion.
